(19) United States
(12) Patent Application Publication
(10) Pub. No.: US 2024/0102086 A1
(43) Pub. Date: Mar. 28, 2024

(54) UNIVERSAL FLUORESCENT PROBES ACTIVATED WITH RNaseH2

(71) Applicant: Bio-Rad Laboratories, Inc., Hercules, CA (US)

(72) Inventors: Séverine MARGERIDON, Hercules, CA (US); Monica HERRERA, Dublin, CA (US); Richard DANNEBAUM, Hercules, CA (US); Nyaradzo DZVOVA, Oakland, CA (US); Raymond-John ABAYAN, Hercules, CA (US); Darren R. LINK, Hercules, CA (US)

(21) Appl. No.: 18/370,566

(22) Filed: Sep. 20, 2023

Related U.S. Application Data
(60) Provisional application No. 63/376,911, filed on Sep. 23, 2022.

(51) Int. Cl. C12Q 1/6853

(52) U.S. Cl. CPC C12Q 1/6853

(57) ABSTRACT

Methods and compositions comprising primers and probes to detect nucleic acids are provided. The probes comprise a ribonucleotide that can be cleaved by an RNase H2 enzyme when the probe is annealed to a reverse complement of a universal sequence that is introduced to a target nucleic acid, for example via amplification.

### CROSS-REFERENCE TO RELATED PATENT APPLICATIONS

The present application claims benefit of priority to U.S. Provisional Patent Application No. 63/376,911, filed on Sep. 23, 2023, which is incorporated by reference for all purposes.

### BACKGROUND OF THE INVENTION

A variety of probe formats have been used to detect amplification products in bulk or in partitions (e.g., dPCR or ddPCR). The most common fluorescent probe used in qPCR (quantitative PCR) and dPCR (digital PCR) are Taqman™ probes that require hydrolysis by the 5′ to 3′ exonuclease activity of the DNA polymerase to generate a fluorescent signal during each amplification signal. Another type of fluorescent probes are molecular beacons, which have a stem-loop structure increasing the binding specificity of the probe to its target. The downside of molecular beacons is that the fluorescence signal produced is not as strong as with hydrolysis probes as the probes are not cleaved throughout the PCR cycles. In the context of dPCR that reads end-point fluorescent signal, molecular beacons hybridize to their targets during a final step consisting of denaturing amplified product (e.g., 10 minutes at 98° C.) followed by a ramp down to 4° C. The default configuration quantifying target DNA with fluorescent hydrolysis probes is to use a primer pair and a fluorescent probe specific to each target amplicon. In the case of multiplex, this means each target amplicon requires a set of 3 components, a forward primer, a reverse primer and a fluorescent probe.

### BRIEF SUMMARY OF THE INVENTION

In some embodiments, methods of detecting a target nucleic acid in a sample are provided. In some embodiments, the method comprises,
(a) forming a reaction mixture comprising:
   sample nucleic acids;
   a plurality of forward primers comprising 3′ target-specific forward sequences,
   a plurality of reverse primers comprising 3′ target-specific reverse sequences, wherein the forward primers or the reverse primers further comprise a 5′ universal sequence;
   probe nucleic acids comprising (i) a fluorophore, (ii) a quencher, (iii) at least 25% (e.g., at least 50%), or at least 10 (e.g., at least 15, 20, 25 or more) nucleotides, or both, of the 5′ universal sequence and (iv) having at least one ribonucleotide separating the fluorophore and the quencher;
   a DNA polymerase; and
   an RNaseH2 enzyme;
(b) annealing the forward primers to target nucleic acids in the sample nucleic acids and extending with a polymerase the forward primers using the target nucleic acids as a template to form first strand extension products;
(c) annealing the reverse primers to the first strand extension products and extending with the polymerase the reverse primers using the first strand extension products as a template to form second strand extension products, wherein the first strand extension products comprise a reverse complement of the 5′ universal sequence if the reverse primers comprise the 5′ universal sequence and the second strand extension products comprise a reverse complement of the 5′ universal sequence if the forward primers comprise the 5′ universal sequence; and
(d) annealing the probe nucleic acids to the reverse complement of the 5′ universal end sequence and the RNase H2 enzyme cleaves the annealed probe at the ribonucleotide, thereby separating the quencher from the fluorophore to generate a detectable signal indicating the presence of the target nucleic acid.

In some embodiments, (a)-(d) occurs in partitions wherein the target nucleic acid is distributed among the partitions such that at least a portion of the partitions do not contain a target nucleic acid.

In some embodiments, the sample nucleic acids are cell-free DNA. In some embodiments, the sample nucleic acids are from a pregnant woman and contain maternal and fetal DNA.

In some embodiments, the partitions are droplets. In some embodiments, the partitions are microwells. In some embodiments, the microwells are sealed by a lid, an air gap, or an oil layer.

In some embodiments, the detectable signal is monitored in real-time. In some embodiments, the detectable signal is monitored at an end point of the method.

In some embodiments, the forward primers comprise the 5′ universal sequence and the concentration of reverse primers is higher than the concentration of forward primers. In some embodiments, the reverse primers comprise the 5′ universal sequence and the concentration of forward primers is higher than the concentration of forward primers.

In some embodiments, the forward primers comprise the 5′ universal sequence and the reverse primers have a 5′ tail sequence that does not anneal to the target nucleic acids. In some embodiments, the reverse primers comprise the 5′ universal sequence and the forward primers have a 5′ tail sequence that does not anneal to the target nucleic acids.

In some embodiments, the probe nucleic acids are linear probes. In some embodiments, the linear probes comprise 80-100% of the 5′ universal sequence. In some embodiments, the probe nucleic acids have at least 10 (e.g., at least 15, 20, 25, 30) nucleotides that anneal to the reverse complement of the 5′ universal sequence.

In some embodiments, the forward primer comprises the 5′ universal sequence and the linear probes and the reverse complement of the 5′ universal sequence on the second strand extension products form a duplex having a higher melting temperature than a duplex formed from the 5′ universal sequence of the forward primer and the reverse complement of the 5′ universal sequence. In some embodiments, the reverse primer comprises the 5′ universal sequence and the linear probes and the reverse complement of the 5′ universal sequence on the first strand extension products form a duplex having a higher melting temperature than a duplex formed from the 5′ universal sequence of the reverse primers and the reverse complement of the 5′ universal sequence.

In some embodiments, the probe nucleic acids form a stem-loop and comprise 5′ to 3′: a first stem sequence, a loop sequence, and a second stem sequence that is the reverse complement of the first stem sequence, wherein the ribonucleotide is in the loop sequence, and wherein the 5′ universal sequence comprises at least part of (or all of) the loop sequence. In some embodiments, the 5′ universal sequence further comprises at least part of (or all of) the second stem sequence. In some embodiments, the first stem sequence and the second stem sequence are each 4-10 (e.g., 4, 5, 6, 7, 8, 9, or 10) nucleotides long. In some embodiments, the loop sequence is between 5-50 (e.g., 10-40 or 17-32) nucleotides long. In some embodiments, the 5′ universal sequence comprises all of the loop sequence, all of the second stem sequence, or all of the loop sequence and second stem sequence.

In some embodiments, the plurality of forward primers comprises at least 2 (e.g., at least 3, 5, 10, 20, 30, 40, 50) different forward primers having 5 different 3′ target-specific sequences and the plurality of reverse primers comprises at least 5 different reverse primers to allow for amplification of 2 (e.g., at least 3, 5, 10, 20, 30, 40, 50) target nucleic acids.

In some embodiments, the RNase H2 enzyme is a Pyrococcus abyssi RNase H2 enzyme or a mutant thereof, Pyrococcus furiosis RNase H2 enzyme or a mutant thereof, Pyrococcus horikoshii RNase H2 enzyme or a mutant thereof, Thermococcus kodakarensis RNase H2 enzyme or a mutant thereof, or a Thermococcus litoralis RNase H2 enzyme or a mutant thereof.

In some embodiments, the reaction mixture comprises:

(a) a first set of the forward and reverse primers, wherein the first set targets a first plurality of different target nucleic acids and the forward or reverse primers of the first set comprise a first 5′ universal sequence, and a first set of the probe nucleic acids comprising (i) a first fluorophore, (ii) a first quencher, (iii) at least 25% (e.g., at least 50%), or at least 10 (e.g., at least 15, 20, 25 or more) nucleotides, or both, of the first 5′ universal sequence; and b) a second set of the forward and reverse primers, wherein the second set targets a second plurality of different target nucleic acids and the forward or reverse primers of the second set comprise a second 5′ universal sequence different from the first 5′ universal sequence, and a second set of the probe nucleic acids comprising (i) a second fluorophore, (ii) a second quencher, (iii) at least 25% (e.g., at least 50%), or at least 10 (e.g., at least 15, 20, 25 or more) nucleotides, or both, of the second 5′ universal sequence, such that first amplicons from the first set of forward and reverse primers and having the first 5′ universal sequence can be distinguished from second amplicons from the second set of forward and reverse primers and having the second 5′ universal sequence based on signal from the first set of the probe nucleic acids and the second set of the probe nucleic acids, respectively.

In some embodiments, the DNA polymerase lacks 5′-3′ exonuclease activity.

Also provided are reaction mixtures. In some embodiments, the reaction mixture comprises
a plurality of forward primers comprising 3′ target-specific forward sequences,
a plurality of reverse primers comprising 3′ target-specific reverse sequences, wherein the forward primers or the reverse primers further comprise a 5′ universal sequence;
probe nucleic acids comprising (i) a fluorophore, (ii) a quencher, (iii) at least 25% (e.g., at least 50%), or at least 10 (e.g., at least 15, 20, 25 or more) nucleotides, or both, of the 5′ universal sequence and (iv) having a least one ribonucleotide separating the fluorophore and the quencher;
a DNA polymerase; and
an RNaseH2 enzyme.

In some embodiments, the reaction mixture further comprises sample nucleic acids. In some embodiments, the sample nucleic acids are cell-free DNA. In some embodiments, the sample nucleic acids are from a pregnant woman and contain maternal and fetal DNA.

In some embodiments, the reaction mixture is in partitions wherein the target nucleic acid is distributed among the partitions such that at least a portion of the partitions do not contain a target nucleic acid. In some embodiments, the partitions are droplets. In some embodiments, the partitions are microwells. In some embodiments, the microwells are sealed by a lid, an air gap, or an oil layer.

In some embodiments, the forward primers comprise the 5′ universal sequence and the concentration of reverse primers is higher than the concentration of forward primers. In some embodiments, the reverse primers comprise the 5′ universal sequence and the concentration of forward primers is higher than the concentration of forward primers.

In some embodiments, the forward primers comprise the 5′ universal sequence and the reverse primers have a 5′ tail sequence that does not anneal to the target nucleic acids. In some embodiments, the reverse primers comprise the 5′ universal sequence and the forward primers have a 5′ tail sequence that does not anneal to the target nucleic acids.

In some embodiments, the probe nucleic acids are linear probes. In some embodiments, the linear probes comprise 80-100% of the 5′ universal sequence. In some embodiments, the probe nucleic acids have at least 10 (e.g., at least 15, 20, 25, 30) nucleotides that anneal to the reverse complement of the 5′ universal sequence.

In some embodiments, the probe nucleic acids form a stem-loop and comprise 5′ to 3′: a first stem sequence, a loop sequence, and a second stem sequence that is the reverse complement of the first stem sequence, wherein the ribonucleotide is in the loop sequence, and wherein the 5′ universal sequence comprises at least part of the loop sequence. In some embodiments, the 5′ universal sequence further comprises at least part of the second stem sequence. In some embodiments, the first stem sequence and the second stem sequence are each 4-10 (e.g., 4, 5, 6, 7, 8, 9, or 10) nucleotides long. In some embodiments, the loop sequence is between 5-50 (e.g., 10-40 or 17-32) nucleotides long. In some embodiments, the 5′ universal sequence comprises all of the loop sequence, all of the second stem sequence, or all of the loop sequence and second stem sequence.

In some embodiments, the plurality of forward primers comprises at least 2 (e.g., at least 3, 5, 10, 20, 30, 40, 50) different forward primers having 5 different 3′ target-specific sequences and the plurality of reverse primers comprises at least 5 different reverse primers to allow for amplification of 2 (e.g., at least 3, 5, 10, 20, 30, 40, 50) target nucleic acids.

In some embodiments, the RNase H2 enzyme is a Pyrococcus abyssi RNase H2 enzyme or a mutant thereof, Pyrococcus furiosis RNase H2 enzyme or a mutant thereof, Pyrococcus horikoshii RNase H2 enzyme or a mutant thereof, Thermococcus kodakarensis RNase H2 enzyme or a mutant thereof, or a Thermococcus litoralis RNase H2 enzyme or a mutant thereof.

In some embodiments, the reaction mixture comprises:
(a) a first set of the forward and reverse primers, wherein the first set targets a first plurality of different target nucleic acids and the forward or reverse primers of the first set comprise a first 5′ universal sequence, and a first set of the probe nucleic acids comprising (i) a first fluorophore,
(ii) a first quencher, (iii) at least 25% (e.g., at least 50%), or at least 10 (e.g., at least 15, 20, 25 or more) nucleotides, or both, of the first 5′ universal sequence; and
(b) a second set of the forward and reverse primers, wherein the second set targets a second plurality of different target nucleic acids and the forward or reverse primers of the second set comprise a second 5′ universal sequence different from the first 5′ universal sequence, and a second set of the probe nucleic acids comprising (i) a second fluorophore, (ii) a second quencher,
(iii) at least 25% (e.g., at least 50%), or at least 10 (e.g., at least 15, 20, 25 or more) nucleotides, or both, of the second 5′ universal sequence, such that first amplicons from the first set of forward and reverse primers and having the first 5′ universal sequence can be distinguished from second amplicons from the second set of forward and reverse primers and having the second 5′ universal sequence based on signal from the first set of the probe nucleic acids and the second set of the probe nucleic acids, respectively.

In some embodiments, the DNA polymerase lacks 5′-3′ exonuclease activity.

Also provided is a mixture comprising,
(a) a first set of forward primers comprising 3′ target-specific forward sequences and reverse primers comprising 3′ target-specific reverse sequences, wherein the forward primers or the reverse primers of the first set further comprise a first 5′ universal sequence, wherein the first set targets a first plurality of different target nucleic acids, and a first set of the probe nucleic acids comprising (i) a first fluorophore, (ii) a first quencher, (iii) at least 25% (e.g., at least 50%), or at least 10 (e.g., at least 15, 20, 25 or more) nucleotides, or both, of the first 5′ universal sequence and (iv) a least one ribonucleotide separating the fluorophore and the quencher; and
(b) a second set of forward primers comprising 3′ target-specific forward sequences and reverse primers comprising 3′ target-specific reverse sequences, wherein the forward primers or the reverse primers of the second set further comprise a second 5′ universal sequence, wherein the second set targets a second plurality of different target nucleic acids, and a second set of the probe nucleic acids comprising (i) a second fluorophore, (ii) a second quencher, (iii) at least 25% (e.g., at least 50%), or at least 10 (e.g., at least 15, 20, 25 or more) nucleotides, or both, of the second 5′ universal sequence and (iv) a least one ribonucleotide separating the fluorophore and the quencher, such that first amplicons from the first set of forward and reverse primers and having the first 5′ universal sequence can be distinguished from second amplicons from the second set of forward and reverse primers and having the second 5′ universal sequence based on signal from the first set of the probe nucleic acids and the second set of the probe nucleic acids, respectively.

In some embodiments, the mixture further comprises an RNaseH2 enzyme.

### BRIEF DESCRIPTION OF THE DRAWINGS

FIG. 1A-D explains the working mechanism of molecular beacon fluorescent probes and the difference when using a molecular beacon with an RNA base in their loop sequence cleaved with RNaseH2 activated fluorescent probes. (1A) During the first cycle of PCR, the 5′ universal tail of the forward primer is incorporated to the amplified DNA template; (1B) During subsequent PCR cycles, reverse strands of DNA comprising the reserve complement of the universal tail sequence incorporated in step A are synthesized. (1C) Standard molecular beacons (without RNA base) bind to the reverse complement of the universal sequence located at the 3′ end of the amplified reverse DNA strands. They bind to their target during annealing steps and are released during denaturation steps. In the context of real-time PCR, the fluorescence signal can be monitored in real time during the annealing step of each PCR cycle. As the quantity of synthesized reverse DNA strand increase, the fluorescence signal increase. In dPCR, the signal is read at end point. To ensure maximum binding of the molecular beacons, a final denaturation step (i.e. 98° C. for 10 min) followed by a ramp down step to 4° C. at 2.5° C./sec may be added. This enables all the amplified DNA molecules and the molecular beacons to fully denature and to find their hybridization targets during the cool down step. Maximum fluorescent signal is generated during that step and remains stable to be read at room temperature. (1D) With Molecular Beacons containing at least 1 RNA base in their loop sequence, the molecular beacons bind to their target during the annealing steps, and once the RNA:DNA hybrid structure is stable, the RNaseH2 cleaves the RNA base of the molecular beacon. This generates a stronger fluorescence signal than just the binding of the molecular beacon itself. New molecular beacons with an RNA base can hybridize and get cleaved during each subsequent cycle of PCR. F=Fluorophore, Q=quencher, R=RNA base.

FIG. 2A-C shows examples of RNaseH2 activated fluorescent probes designs. (2A) The 5′ universal tail of the forward primer comprises the same sequence as the loop sequence of the molecular beacon. One base at the center of the molecular beacon loop is changed from a DNA to an RNA base. During annealing steps, the loop sequence of the molecular beacon binds to the reverse complement of the universal sequence, and the RNA base is cleaved by the RnaseH2. (2B) The 5′ universal tail of the forward primer can comprise the same sequence as the loop sequence and one of the stem sequences of the molecular beacon. One base at the center of the molecular beacon loop is changed from a DNA to an RNA base. During annealing steps, the loop sequence and one stem sequence of the molecular beacon bind to the reverse complement of the universal sequence, and the RNA base is cleaved by the RnaseH2. (2C) The 5′ universal tail of the forward primer can comprise the full length of a linear probe sequence. One base at the center of the linear probe is changed from a DNA to an RNA base. During annealing steps, the full length of the linear probe binds to the reverse complement of the universal sequence, and the RNA base is cleaved by the RnaseH2. F=Fluorophore, Q=quencher, R=RNA base.

FIG. 3. Differences in fluorescence separation using molecular beacon probe and linear probe configurations with RNA base tested with and without RNase H2.

FIG. 4 displays results from a 60-plex tested on the Bio-Rad QX600 Droplet Digital PCR System comparing standard molecular beacons and RNaseH2 cleavable molecular beacons.

### DEFINITIONS

Unless defined otherwise, all technical and scientific terms used herein generally have the same meaning as commonly understood by one of ordinary skill in the art to which this disclosure belongs. Generally, the nomenclature used herein and the laboratory procedures in cell culture, molecular genetics, organic chemistry, and nucleic acid chemistry and hybridization described below are those well-known and commonly employed in the art. Standard techniques are used for nucleic acid and peptide synthesis. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document. The nomenclature used herein and the laboratory procedures in analytical chemistry, and organic synthetic described below are those well-known and commonly employed in the art.

The term “amplification” refers to any in vitro means for multiplying the copies of a target sequence of nucleic acid in a linear or exponential manner. Such methods include but are not limited to polymerase chain reaction (PCR).

“Amplifying” refers to a step of submitting a solution to conditions sufficient to allow for amplification of a polynucleotide if all of the components of the reaction are intact. Components of an amplification reaction include, e.g., primers, a polynucleotide template, polymerase, nucleotides, and the like. The term “amplifying” typically refers to an “exponential” increase in target nucleic acid. However, “amplifying” as used herein can also refer to linear increases in the numbers of a select target sequence of nucleic acid, such as is obtained with cycle sequencing or linear amplification. In an exemplary embodiment, amplifying refers to PCR amplification using a first and a second amplification primer.

The term “amplification reaction mixture” refers to an aqueous solution comprising the various reagents used to amplify a target nucleic acid. These include enzymes, aqueous buffers, salts, amplification primers, target nucleic acid, and nucleoside triphosphates. Amplification reaction mixtures may also further include stabilizers and other additives to optimize efficiency and specificity.

“Polymerase chain reaction” or “PCR” refers to a method whereby a specific segment or subsequence of a target double-stranded DNA, is amplified in a geometric progression. PCR is well known to those of skill in the art; see, e.g., U.S. Pat. Nos. 4,683,195 and 4,683,202; and PCR Protocols: A Guide to Methods and Applications, Innis et al., eds, 1990. Exemplary PCR reaction conditions typically comprise either two or three step cycles. Two step cycles have a denaturation step followed by a hybridization/elongation step. Three step cycles comprise a denaturation step followed by a hybridization step followed by a separate elongation step.

A “primer” refers to a polynucleotide sequence that hybridizes to a sequence on a target nucleic acid and serves as a point of initiation of nucleic acid synthesis. Primers can be of a variety of lengths and in some embodiments are less than 60 nucleotides in length, for example 12-35 nucleotides, in length. The length and sequences of primers for use in PCR can be designed based on principles known to those of skill in the art, see, e.g., Innis et al., supra. Primers can be DNA, RNA, or a chimera of DNA and RNA portions. In some cases, primers can include one or more modified or non-natural nucleotide bases. In some cases, primers are labeled.

The term “probe” refers to a nucleic acid that changes signal status depending on whether the probe anneals to a target nucleic acid or not, as described herein. The probe nucleic acid, in some embodiments, comprises a blocked 3′ end preventing a polymerase from extending the annealed probe.

A “template” or “target” refers to a polynucleotide sequence that comprises the polynucleotide to be amplified, flanked by one or a pair of primer hybridization sites. Thus, a “target nucleic acid” comprises the target polynucleotide sequence adjacent to at least one hybridization site for a primer. In some cases, a “target nucleic acid” comprises the target polynucleotide sequence flanked by a hybridization site for a “forward” primer and a “reverse” primer.

As used herein, “nucleic acid” means DNA, RNA, single-stranded, double-stranded, or more highly aggregated hybridization motifs, and any chemical modifications thereof. Modifications include, but are not limited to, those providing chemical groups that incorporate additional charge, polarizability, hydrogen bonding, electrostatic interaction, points of attachment and functionality to the nucleic acid ligand bases or to the nucleic acid ligand as a whole. Such modifications include, but are not limited to, peptide nucleic acids (PNAs), Locked nucleic acids (LNAs)(see, e.g., WO99/14226, WO98/39352, WO2003/-20739, U.S. Pat. No. 7,053,207), phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases, isocytidine and isoguanidine and the like. Nucleic acids can also include non-natural bases, such as, for example, nitroindole. Modifications can also include 3′ and 5′ modifications including but not limited to capping with a fluorophore (e.g., quantum dot) or another moiety.

A “polymerase” refers to an enzyme that performs template-directed synthesis of polynucleotides, e.g., DNA and/or RNA. The term encompasses both the full length polypeptide and a domain that has polymerase activity. DNA polymerases are well-known to those skilled in the art, including but not limited to DNA polymerases isolated or derived from Pyrococcus furiosus, Thermococcus litoralis, and Thermotoga maritime, or modified versions thereof. Additional examples of commercially available polymerase enzymes include, but are not limited to: Klenow fragment (New England Biolabs® Inc.), Taq DNA polymerase (QIAGEN), 9° N™ DNA polymerase (New England Biolabs® Inc.), Deep Vent™ DNA polymerase (New England Biolabs® Inc.), Manta DNA polymerase (Enzymatics®), Bst DNA polymerase (New England Biolabs® Inc.), and phi29 DNA polymerase (New England Biolabs® Inc.). At least five families of DNA-dependent DNA polymerases are known, although most fall into families A, B and C. Other types of DNA polymerases include phage polymerases.

As used herein, the term “partitioning” or “partitioned” refers to separating a sample into a plurality of portions, or “partitions.” Partitions are generally physical, such that a sample in one partition does not, or does not substantially, mix with a sample in an adjacent partition. Partitions can be solid or fluid. In some embodiments, a partition is a solid partition, e.g., a microchannel. In some embodiments, a partition is a fluid partition, e.g., a droplet. In some embodiments, a fluid partition (e.g., a droplet) is a mixture of immiscible fluids (e.g., water and oil). In some embodiments, a fluid partition (e.g., a droplet) is an aqueous droplet that is surrounded by an immiscible carrier fluid (e.g., oil).

### DETAILED DESCRIPTION OF THE INVENTION

Methods of detecting target nucleic acids are provided using probes having a ribonucleotide. The probes are cleaved by a RNaseH2 enzyme when bound to a universal sequence introduced to a target nucleic acid by a primer. For example, the probes described herein can have a fluorophore and quencher moiety separated by the ribonucleotide such that signal from the fluorophore in an intact probe is relatively quenched compared to when the probe is cleaved at the ribonucleotide by the RNaseH2 enzyme. The inventors have found that such probes are of particular use in digital amplification assays and also will be useful in real-time bulk amplification assays. Some advantages of these methods include better separation of signal (target) from background (no target) as well as the ability to use DNA polymerases lacking 5′-3′ exonuclease activity. The methods described herein can also be used in various multiplex formats allowing for detection of large numbers of different amplicons as desired. Additional advantages will be apparent from the remainder of this disclosure.

The methods can involve formation of a reaction mixture comprising sample nucleic acids, forward and reverse primers, probes as described herein, a DNA polymerase and a RNaseH2 enzyme. Each of these will be discussed in turn followed by a discussion of methods of their use.

A target nucleic acid is any DNA sequence (e.g., dsDNA, cDNA) to be detected from a sample. Biological samples can be obtained from any biological organism, e.g., an animal, plant, fungus, pathogen (e.g., bacteria or virus), or any other organism. In some embodiments, the biological sample is from an animal, e.g., a mammal (e.g., a human or a non-human primate, a cow, horse, pig, sheep, cat, dog, mouse, or rat), a bird (e.g., chicken), or a fish. A biological sample can be any tissue or bodily fluid obtained from the biological organism, e.g., blood, a blood fraction, or a blood product (e.g., serum, plasma, platelets, red blood cells, and the like), sputum or saliva, tissue (e.g., kidney, lung, liver, heart, brain, nervous tissue, thyroid, eye, skeletal muscle, cartilage, or bone tissue); cultured cells, e.g., primary cultures, explants, and transformed cells, stem cells, stool, urine, etc. In some embodiments, the sample is a cell-free nucleic acid sample. In some embodiments, the cell-free DNA sample is from a pregnant woman (for example, a cell-free sample from maternal blood containing maternal and fetal DNA) or a person having, or who had or is suspected of having cancer or who is an organ transplant recipient.

The reaction mixture will have a plurality of forward primers comprising one or more 3′ target-specific forward sequences. The terms “forward” and “reverse” are arbitrary designations, indicating that forward and reverse primers anneal to opposite strands of a double-stranded DNA molecule and when annealed have 3′ ends directed towards each other such that an amplicon is formed under PCR conditions. The 3′ target-specific forward sequences can vary in composition and length and can be designed to specifically amplify a particular target nucleic acid in a mixture of different nucleic acids. In some embodiments, the 3′ target-specific forward sequence is between 10-30 nucleotides long that anneals to or adjacent to a target nucleic acid though other lengths can be used in other embodiments.

The plurality of forward primers will include a number of copies of the primers to achieve the desired amplification. In multiplex reactions, the plurality of forward primers will further include multiple different forward primers having different 3′ target-specific forward sequences, allowing for amplification of different targets in the same reaction. As a simple example, the reaction can have copies of a first forward primer having a first 3′ target-specific forward sequence and copies of a second forward primer having a second (different from first) 3′ target-specific forward sequence, allowing for amplification of a first and second target, if present in the sample. As discussed more below, the number of different forward primers can be high, e.g., at least 2, 5, 10, 20, 30, 40, 50, 70, 100, 120, or more, for example, from 2-200, in the same reaction. As will be discussed below, in some embodiments, the universal sequence will be located on the forward primers and will be located at the 5′ end of the forward primers in this case.

In some embodiments, a plurality of reverse primers is provided. For example, in some embodiments, for each forward primer having a unique 3′ target-specific forward sequence there will be one reverse primer with a unique 3′ target-specific reverse sequence such that the forward and reverse primer together amplify a particular target nucleic acid. As with the forward primer, in multiplex options, reverse primers with different 3′ target-specific reverse sequences can be employed in the reaction mixture.

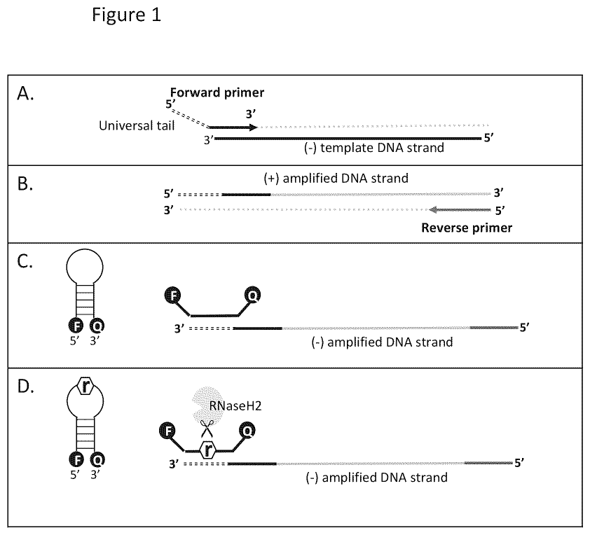

Either the forward primers or the reverse primers, but in some embodiments not both, will further comprise a 5′ universal sequence. The 5′ universal sequence is a sequence common to a set of primers but does not anneal to the target sequence in the initial amplification round (they are not target-specific). See, e.g., FIG. 1. The 5′ universal sequence can be any length as desired so long as it is of sufficient length for annealing of the probe during the reaction as discussed below. In some embodiments, for example the 5′ universal sequence is between 10-50 nucleotides long, e.g., 15-40, 15-35, or 20-35 nucleotides long. At least a part of, and in some embodiments, all of, the 5′ universal primer, and at least part of the probe sequence, including the ribonucleotide(s) of the probe, will have identical sequences to each other such that the probe, including the ribonucleotide(s) and the reverse complement of the universal sequence will anneal, allowing for cleavage at the ribonucleotide by the RNaseH2 enzyme as explained further below.

In embodiments in which a forward and reverse primer pair is provided, it can be advantageous to include a high number (e.g., high concentration) of the primer that does not have the 5′ universal sequence compared to the primer that does have the 5′ universal sequence. For example, in embodiments in which the forward primer has the 5′ universal sequence, in some cases the concentration of the reverse primer will be higher (e.g., at least 1.5, 2, 3, 5, 10 times higher) than the concentration of the forward primer. In embodiments in which the reverse primer has the 5′ universal sequence, in some cases the concentration of the forward primer will be higher (e.g., at least 1.5, 2, 3, 5, 10 times higher) than the concentration of the reverse primer. As an example, in some embodiments, the primer lacking the 5′ universal sequence has a concentration of 500-2000 nM (e.g., 900 or 1000 nM) and the primer having the 5′ universal sequence is provided at a concentration of 50-200 nM (e.g., 90 or 100 nM). This is useful for generating an excess of the strand having the reverse complement of the 5′ universal sequence, which is detected by the probe.

In some multiplex embodiments, some primers can have different 5′ universal sequences, allowing for use of different “color” probes in the same reaction. For example, in some embodiments, a first set of primers (forward or reverse) have a first 5′ universal sequence and a second set of primers (forward or reverse) have a second 5′ universal sequence. In these embodiments, the reaction mixture has a first probe with a sequence identical to part or all of the first 5′ universal sequence and a second probe with a sequence identical to part or all of the second 5′ universal sequence. If the first and second probes have different linked fluorophores or quenchers such that the signals emitted have a different wavelength (color) then the first and second probe signals can be distinguished. While the example above is illustrated with two 5′ universal sequences and associated probes, one can employ more (e.g., 3, 4, 5, 6, 7, 8, 9, 10, etc.) 5′ universal sequences and associated probes wherein each probe can be distinguished from the other by the wavelength of their signals.

Moreover, in some embodiments, each set of primers having the same 5′ universal sequence can include primers with more than one different 3′ target-specific forward (or reverse depending on which primer) sequence. Thus for example, a first set of forward primers could include 2, 5, 10, or 20, or more different 3′ target-specific forward sequences and each have a first 5′ universal sequence, and thus would be detectable by a first probe and a second set of forward primers could include 2, 5, 10, or 20, or more different 3′ target-specific forward sequences and each have a second 5′ universal sequence, and thus would be detectable by a second probe. When all in one reaction mixture, this allows for a very high number of different amplifications to be monitored. For example, the inventors have used six distinguishable probes, each set probing 20 different amplification reactions having the set's own 5′ universal sequence, allowing for 120 different reactions to be monitored in digital format.

Probe nucleic acids use energy transfer between two moieties, e.g., a donor fluorophore and an acceptor moiety separated by the ribonucleotide. In some embodiments, the probe nucleic acids comprise a fluorophore moiety and quencher moiety (i.e., an acceptor that absorbs energy released by the donor fluorophore, but then does not itself fluoresce) separated by the ribonucleotide such that upon cleavage of the ribonucleotide, the two moieties are separated, resulting in a detectable signal. The fluorophore and quencher can be located at the ends of the probe, respectively, or one or both can be linked to an internal nucleotide in the probe. In some embodiments, the proximity of the quencher to the fluorophore in the intact probe results in quenched fluorescent signal, whereas the cleaved probe results in increased detectable signal due to a reduction or lack of quenching. In some embodiments in which two or more different probes are used, the different probes will have a different fluorophore or quencher or both such that the different probes emit signal at different wavelengths that can be differentiated from each other by a sensor. Because some quenchers can quench a broad range of wavelengths, in some embodiments, the same quencher is used for two or more different probes, each of which have different fluorophores that emit at different wavelengths.

Fluorescent agents can include a variety of organic and/or inorganic small molecules or a variety of fluorescent proteins and derivatives thereof. A vast array of fluorophores and quenchers are reported in the literature and thus known to those skilled in the art, and many are readily available from commercial suppliers to the biotechnology industry. Literature sources for fluorophores include Cardullo et al., Proc. Natl. Acad. Sci. USA 85: 8790-8794 (1988); Dexter, D. L., J. of Chemical Physics 21: 836-850 (1953); Hochstrasser et al., Biophysical Chemistry 45: 133-141 (1992); Selvin, P., Methods in Enzymology 246: 300-334 (1995); Steinberg, I. Ann. Rev. Biochem., 40: 83-114 (1971); Stryer, L. Ann. Rev. Biochem., 47: 819-846 (1978); Wang et al., Tetrahedron Letters 31: 6493-6496 (1990); Wang et al., Anal. Chem. 67: 1197-1203 (1995). Non-limiting examples of fluorophores include cyanines, fluoresceins (e.g., 5′-carboxyfluorescein (FAM), Oregon Green, and Alexa 488), HEX, rhodamines (e.g., N,N,N′,N′-tetramethyl-6-carboxyrhodamine (TAMRA), tetramethyl rhodamine, and tetramethyl rhodamine isothiocyanate (TRITC)), eosin, coumarins, pyrenes, tetrapyrroles, arylmethines, oxazines, polymer dots, and quantum dots. In some embodiments, the fluorophores are selected from HEX, FAM, Cy5, Cy5.5, ROX, Atto 590, Alexa 405, Pacific Blue, ABY, and Texas Red. Exemplary quenchers can include, but are not limited to, Iowa Black® FQ, Iowa Black® RQ, Black Hole Quencher®-1, Black Hole Quencher®-2, DABCYL, and ZEN internal quencher.

The nucleotide sequence of the probe (including the position of the ribonucleotide) will comprise at least 25%, and more preferably, at least 30, 40, 50, 60, 70, 80, 90, or 100% of the 5′ universal sequence. For example, in some embodiments, the probe can comprise 10-50, e.g., 15-30 nucleotides identical to the 5′ universal sequence. At the aligning position, the ribonucleotide will include the same nucleotide (but in ribonucleotide form) as the 5′ universal sequence, except if the position in the 5′ universal sequence is thymine, the ribonucleotide is the RNA equivalent, i.e., uracil. The nucleotide sequence will be composed of non-ribonucleotides aside from the one or more ribonucleotide separating the fluorophore and quencher moiety. The non-ribonucleotides can be for example deoxyribonucleotides (e.g., forming DNA) or analogs thereof. In some embodiments, the probe can include one or more non-natural nucleotide or analog thereof (e.g., peptide-nucleic acids (PNAs) or Locked nucleic acids (LNAs)) or other nucleotides that have greater affinity for the reverse complement of the 5′ universal sequence compared to the primer having the 5′ universal sequence (composed of DNA). Increased affinity (high melting temperature (Tm)) of the probe for the reverse complement of the 5′ universal sequence, compared to the primer having the 5′ universal sequence for the reverse complement of the 5′ universal sequence, can be used to further improve signal-to-noise differentiation.

As noted above, one or more ribonucleotide is positioned in the probe between the fluorophore and quencher such that when the RNaseH2 enzyme cleaves at the ribonucleotide, the fluorophore and quencher will diffuse from each other to generate the detectable signal. Moreover, the RNaseH2 enzyme cleaves ribonucleotides annealed to a deoxyribonucleotide counterpart in the context of two anneal strands (in this case the probe and reverse complement of the 5′ universal sequence). RNaseH2 enzyme cleavage is more efficient if there are multiple (e.g., 2, 3, 4, 5, 6, or more) annealed nucleotides on both sides of the ribonucleotide. Thus, in some embodiments, the ribonucleotide is positioned in a middle section of the probe or at least more than 2, 3, 4, 5, 6 or more nucleotides from either end of the probe.

Optionally the probe will be modified at the 3′ end so that a polymerase cannot extend the probe. In some embodiments, the fluorophore or quencher is linked to the 3′ end of the probe and this blocks the polymerase from extending the probe. In other embodiments, the 3′ end of the probe can be modified to block extension. Non-limiting modifications of the 3′ end can include, for example, a 3′ inverted dT, a 3′ C3 spacer, a 3′ amino, or 3′ phosphorylation.

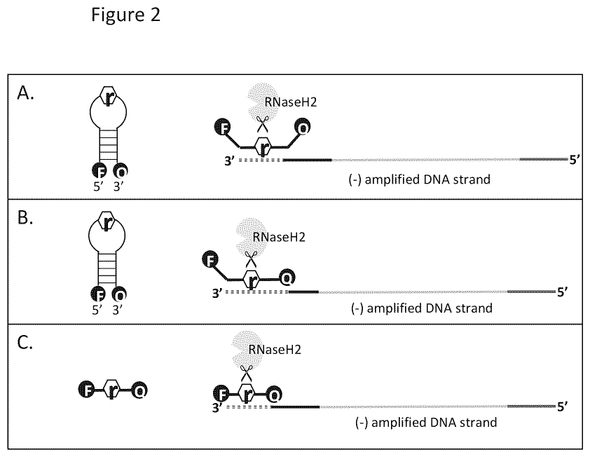

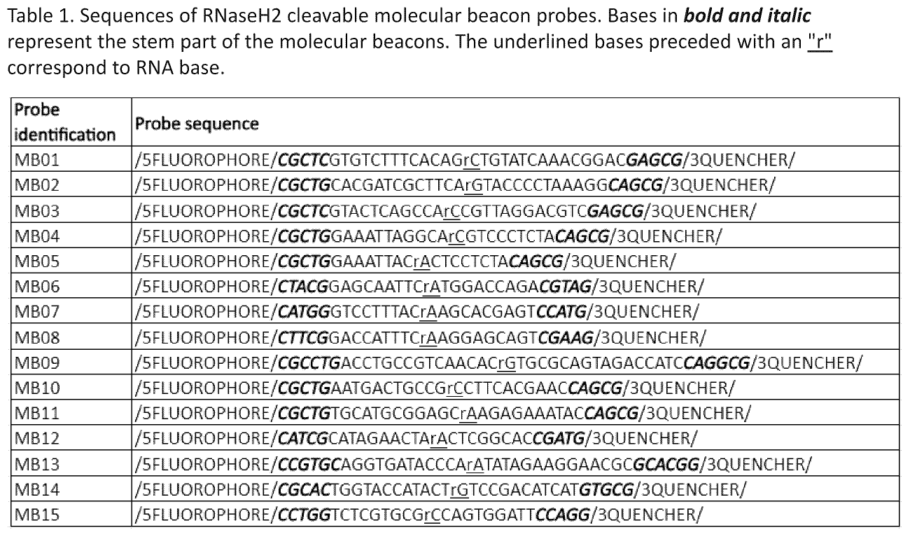

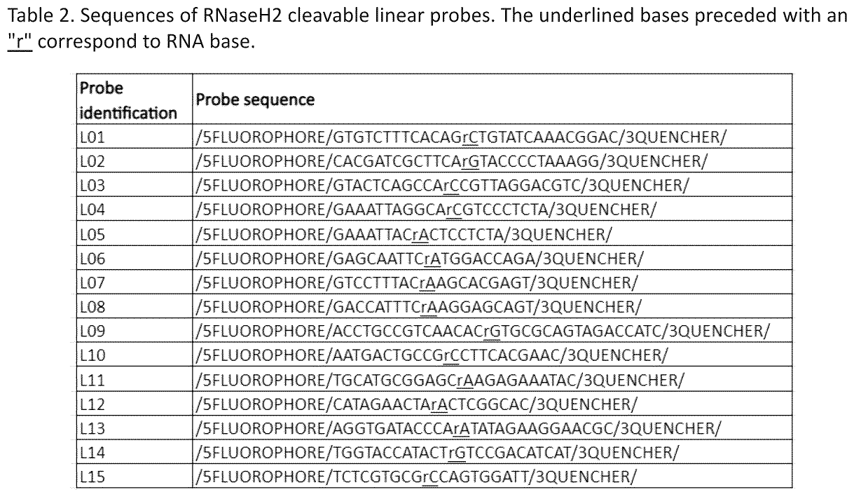

Various probe formats can be used. In some embodiments, the probe is a linear probe, meaning that the sequences within the probe do not form a stem-loop structure or otherwise self-anneal. In other embodiments, the probe can be in a stem-loop format where the ribonucleotide is located in the loop portion. See, e.g., FIG. 2. Probe concentration can be selected to avoid background while generating detectable signal. For example, in some embodiments, the probe is provided at a concentration between 100-1000 nM, e.g., 500 nM. In some embodiments, the probe concentration is lower than the limiting primer (i.e., the primer having the 5′ universal sequence) concentration, and in some embodiments, is 2-10 times the limiting primer concentration.

Linear probes can be of any length as desired. Exemplary probe nucleic acids can have, for example, at least 10 (e.g., at least 15, 20, 25, 30) contiguous or non-contiguous nucleotides that are reverse complementary to the 5′ universal sequence. In some embodiments, the linear probe will comprise a sequence at least 80, 90, or 100% identical to the 5′ universal probe sequence, thereby improving the probe's ability to compete for the reverse complement of the universal 5′ sequence compared to the primer having the 5′ universal sequence.

Stem-loop probes will have a first stem sequence, a loop sequence, and a second stem sequence that is the reverse complement of the first stem sequence, allowing for the two stem sequences to self-anneal to form a stem-loop. At least part of the loop sequence, which comprises the ribonucleotide, will have the same sequence as the 5′ universal sequence. In some embodiments, at least part of the second stem sequence is also the same as a sequence in the 5′ universal sequence. And in some embodiments, all of the loop or second stem sequence or both are identical to adjacent sequences in the 5′ universal sequence. See, e.g., FIG. 2.

The lengths of the stem sequences and the loop sequences can be varied as desired. In some embodiments, the first stem sequence and the second stem sequence are each 4-10 (e.g., 4, 5, 6, 7, 8, 9, or 10) nucleotides long though other lengths can also be employed. In some embodiments, the loop sequence is between 5-50 (e.g., 10-40 or 17-32) nucleotides long though other lengths can also be employed

The reaction mixtures can comprise a DNA polymerase that acts to extend at least one of the primers in a template-dependent manner. In some embodiments, the DNA polymerase is a thermostable polymerase. Thermostable polymerases are isolated from a wide variety of thermophilic bacteria, such as Thermus aquaticus (Taq), Pyrococcus furiosus (Pfu), Pyrococcus woesei (Pwo), Bacillus sterothermophilus (B st), Sulfolobus acidocaldarius (Sac) Sulfolobus solfataricus (Sso), Pyrodictium occultum (Poc), Pyrodictium abyssi (Pab), and Methanobacterium thermoautotrophicum (Mth), as well as other species. DNA polymerases are known in the art and are commercially available. In some embodiments, the DNA polymerase is Taq, Tbr, Tfl, Tru, Tth, Tli, Tac, Tne, Tma, Tih, Tfi, Pfu, Pwo, Kod, Bst, Sac, Sso, Poc, Pab, Mth, Pho, ES4, VENT™, DEEPVENT™, or an active mutant, variant, or derivative thereof. In some embodiments, the DNA polymerase is Taq DNA polymerase. In some embodiments, the DNA polymerase is a high fidelity DNA polymerase (e.g., iProofrM High-Fidelity DNA Polymerase, Phusion® High-Fidelity DNA polymerase, Q5® High-Fidelity DNA polymerase, Platinum® Taq High Fidelity DNA polymerase, Accura® High-Fidelity Polymerase). In some embodiments, the DNA polymerase is a fast-start polymerase (e.g., FastStart™ Taq DNA polymerase or FastStart™ High Fidelity DNA polymerase). In some embodiments, the polymerase lacks 5′-3′ exonuclease activity, for example, as found in Family B polymerases.

As noted above, the methods and compositions can involve the activity of a RNaseH2 enzyme. A RNaseH2 enzyme cleaves a ribonucleotide in an RNA/DNA duplex. A variety of RNAseH2 enzymes are known and can be used in the methods described herein. Exemplary RNAseH2 enzymes have been described, for example from Pyrococcus abyssi, Pyrococcus furiosis, Pyrococcus horikoshii, Thermococcus kodakarensis, and Thermococcus litoralis. In addition, a variety of mutant enzymes have been developed from these natural enzymes, for example, as described in WO2018/031625, which is incorporated by reference.

The methods herein comprise forming a reaction mixture with some of all of the components described above, as well as reagents necessary for primer extension (e.g., amplification).

The amplification reaction mixture will also comprise nucleotides. Nucleotides for use in the methods described herein can be any nucleotide useful in the polymerization of a nucleic acid. Nucleotides can be naturally occurring, unusual, modified, derivative, or artificial. Nucleotides will generally be unlabeled in the embodiments described herein.

In some embodiments, the amplification reaction mixture comprises one or more buffers or salts. A wide variety of buffers and salt solutions and modified buffers are known in the art. For example, in some embodiments, the buffer is TRIS, TRICINE, BIS-TRICINE, HEPES, MOPS, TES, TAPS, PIPES, or CAPS. In some embodiments, the salt is potassium acetate, potassium sulfate, potassium chloride, ammonium sulfate, ammonium chloride, ammonium acetate, magnesium chloride, magnesium acetate, magnesium sulfate, manganese chloride, manganese acetate, manganese sulfate, sodium chloride, sodium acetate, lithium chloride, or lithium acetate. In some embodiments, the amplification reaction mixture comprises a salt (e.g., potassium chloride) at a concentration of about 10 mM to about 100 mM.

In some embodiments, the amplification reaction mixture comprises one or more stabilizers. Stabilizers for use in the methods described herein include, but are not limited to, polyol (glycerol, threitol, etc.), a polyether including cyclic polyethers, polyethylene glycol, organic or inorganic salts, such as ammonium sulfate, sodium sulfate, sodium molybdate, sodium tungstate, organic sulfonate, etc., sugars, polyalcohols, amino acids, peptides or carboxylic acids, a quencher and/or scavenger such, as mannitol, glycerol, reduced glutathione, superoxide dismutase, bovine serum albumin (BSA) or gelatine, spermidine, dithiothreitol (or mercaptoethanol) and/or detergents such as TRITON® X-100

[Octophenol(ethyleneglycolether)], THESIT® [Polyoxyethylene 9 lauryl ether (Polidocanol C₁₂ E₉)], TWEEN® (Polyoxyethylenesorbitan monolaurate 20, NP40) and BRIJ®-35 (Polyoxyethylene23 lauryl ether).

Once formed, the reaction mixture is submitted to conditions to allow for primer extension using one or more primer that anneals to a target nucleic acid to be detected. The reaction mixture is submitted to primer extension conditions, which can be but is not limited to PCR conditions, allowing the primer to anneal to the target nucleic acid, if present, and being extended by a polymerase in a template (i.e., target nucleic acid)-specific manner. The reaction can be performed in bulk, or in partitions, and can be monitored for signal at an end-point or in real time, i.e., continuously or every cycle, for example.

In embodiments comprising a forward and reverse primer, the forward primer is extended when the target nucleic acid is present, to form a first strand, which is followed by extension of the reverse primer using the first strand as a template to form a second strand complementary to the first strand. Depending on whether the 5′ universal sequence is on the forward or reverse primers, the first or second strand will comprise the 5′ universal strand, respectively, and therefore the second or first strand, respectively, will comprise the reverse complement of the 5′ universal sequence. Because the probe nucleic acids and RNase H2 enzyme are also in the reaction mixture, as the reverse complement of the 5′ universal sequence is generated, the probe will anneal to the reverse complement of the 5′ universal sequence and the annealed probe will be cleaved at the ribonucleotide by the RNase H2 enzyme, separating the fluorophore and the quencher, resulting in a detectable fluorescent signal. The quantity of this signal can indicate the quantity or at least presence of the target nucleic acid in a bulk reaction, or alternatively, if the reaction is in partitions, the number of partitions having a signal above a threshold will be proportional to the quantity of the target nucleic acid in the sample. In general, the amount of sample nucleic acid and number of partitions is selected such that at least some partitions are empty as dictated by a Poisson distribution.

Methods and compositions for partitioning a sample are described, for example, in published patent applications WO 2010/036,352, US 2010/0173,394, US 2011/0092,373, and US 2011/0092,376, the contents of each of which are incorporated herein by reference in the entirety. The plurality of mixture partitions can be in a plurality of emulsion droplets, or a plurality of microwells, etc.

In some embodiments, sample nucleic acids can be partitioned into a plurality of mixture partitions, and then one or more amplification primer(s), probe(s), enzyme(s), oligonucleotides or a combination thereof, can be introduced into the plurality of mixture partitions. Methods and compositions for delivering reagents to one or more mixture partitions include microfluidic methods as known in the art; droplet or microcapsule merging, coalescing, fusing, bursting, or degrading (e.g., as described in U.S. 2015/0027,892; US 2014/0227,684; WO 2012/149,042; and WO 2014/028,537); droplet injection methods (e.g., as described in WO 2010/151,776); and combinations thereof.

The mixture partitions can be picowells, nanowells, or microwells. The mixture partitions can be pico-, nano-, or micro-reaction chambers, such as pico, nano, or microcapsules. The mixture partitions can be pico-, nano-, or micro-channels. The mixture partitions can be droplets, e.g., emulsion droplets.

In some embodiments, the sample nucleic acids and PCR reaction components are partitioned into a plurality of droplets. In some embodiments, a droplet comprises an emulsion composition, i.e., a mixture of immiscible fluids (e.g., water and oil). In some embodiments, a droplet is an aqueous droplet that is surrounded by an immiscible carrier fluid (e.g., oil). In some embodiments, a droplet is an oil droplet that is surrounded by an immiscible carrier fluid (e.g., an aqueous solution). In some embodiments, the droplets are relatively stable and have minimal coalescence between two or more droplets. In some embodiments, less than 0.0001%, 0.0005%, 0.001%, 0.005%, 0.01%, 0.05%, 0.1%, 0.5%, 1%, 2%, 3%, 4%, 5%, 6%, 7%, 8%, 9%, or 10% of droplets generated from a sample coalesce with other droplets. The emulsions can also have limited flocculation, a process by which the dispersed phase comes out of suspension in flakes. Methods of emulsion formation are described, for example, in published patent applications WO 2011/109546 and WO 2012/061444, the entire content of each of which is incorporated by reference herein.

In some embodiments, the droplet is formed by flowing an oil phase through an aqueous sample comprising the sample and reaction components. The oil phase may comprise a fluorinated base oil which may additionally be stabilized by combination with a fluorinated surfactant such as a perfluorinated polyether. In some embodiments, the base oil comprises one or more of a HFE 7500, FC-40, FC-43, FC-70, or another common fluorinated oil. In some embodiments, the oil phase comprises an anionic fluorosurfactant. In some embodiments, the anionic fluorosurfactant is Ammonium Krytox (Krytox-AS), the ammonium salt of Krytox FSH, or a morpholino derivative of Krytox FSH. Krytox-AS may be present at a concentration of about 0.1%, 0.2%, 0.3%, 0.4%, 0.5%, 0.6%, 0.7%, 0.8%, 0.9%, 1.0%, 2.0%, 3.0%, or 4.0% (w/w). In some embodiments, the concentration of Krytox-AS is about 1.8%. In some embodiments, the concentration of Krytox-AS is about 1.62%. Morpholino derivative of Krytox FSH may be present at a concentration of about 0.1%, 0.2%, 0.3%, 0.4%, 0.5%, 0.6%, 0.7%, 0.8%, 0.9%, 1.0%, 2.0%, 3.0%, or 4.0% (w/w). In some embodiments, the concentration of morpholino derivative of Krytox FSH is about 1.8%. In some embodiments, the concentration of morpholino derivative of Krytox FSH is about 1.62%.

In some embodiments, the oil phase further comprises an additive for tuning the oil properties, such as vapor pressure, viscosity, or surface tension. Non-limiting examples include perfluorooctanol and 1H,1H,2H,2H-Perfluorodecanol. In some embodiments, 1H,1H,2H,2H-Perfluorodecanol is added to a concentration of about 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.25%, 1.50%, 1.75%, 2.0%, 2.25%, 2.5%, 2.75%, or 3.0% (w/w). In some embodiments, 1H,1H,2H,2H-Perfluorodecanol is added to a concentration of about 0.18% (w/w).

In some embodiments, the emulsion is formulated to produce highly monodisperse droplets having a liquid-like interfacial film that can be converted by heating into microcapsules having a solid-like interfacial film; such microcapsules may behave as bioreactors able to retain their contents through an incubation period. See, e.g., U.S. Pat. No. 10,378,048. The conversion to microcapsule form may occur upon heating. For example, such conversion may occur at a temperature of greater than about 40°, 50°, 60°, 70°, 80°, 90°, or 95° C. During the heating process, a fluid or mineral oil overlay may be used to prevent evaporation. Excess continuous phase oil may or may not be removed prior to heating. The biocompatible capsules may be resistant to coalescence and/or flocculation across a wide range of thermal and mechanical processing. Following conversion, the microcapsules may be stored at about −70°, −20°, 0°, 3°, 4°, 5°, 6°, 7°, 8°, 9°, 10°, 15°, 20°, 25°, 30°, 35°, or 40° C.

The microcapsule partitions, which may contain one or more polynucleotide sequences and/or one or more sets of primers, may resist coalescence, particularly at high temperatures. Accordingly, the capsules can be incubated at a very high density (e.g., number of partitions per unit volume). In some embodiments, greater than 100,000, 500,000, 1,000,000, 1,500,000, 2,000,000, 2,500,000, 5,000,000, or 10,000,000 partitions may be incubated per mL. In some embodiments, the sample-probe incubations occur in a single well, e.g., a well of a microtiter plate, without inter-mixing between partitions. The microcapsules may also contain other components necessary for the incubation.

In some embodiments, a sample is partitioned into at least 500 partitions, at least 1000 partitions, at least 2000 partitions, at least 3000 partitions, at least 4000 partitions, at least 5000 partitions, at least 6000 partitions, at least 7000 partitions, at least 8000 partitions, at least 10,000 partitions, at least 15,000 partitions, at least 20,000 partitions, at least 30,000 partitions, at least 40,000 partitions, at least 50,000 partitions, at least 60,000 partitions, at least 70,000 partitions, at least 80,000 partitions, at least 90,000 partitions, at least 100,000 partitions, at least 200,000 partitions, at least 300,000 partitions, at least 400,000 partitions, at least 500,000 partitions, at least 600,000 partitions, at least 700,000 partitions, at least 800,000 partitions, at least 900,000 partitions, at least 1,000,000 partitions, at least 2,000,000 partitions, at least 3,000,000 partitions, at least 4,000,000 partitions, at least 5,000,000 partitions, at least 10,000,000 partitions, at least 20,000,000 partitions, at least 30,000,000 partitions, at least 40,000,000 partitions, at least 50,000,000 partitions, at least 60,000,000 partitions, at least 70,000,000 partitions, at least 80,000,000 partitions, at least 90,000,000 partitions, at least 100,000,000 partitions, at least 150,000,000 partitions, or at least 200,000,000 partitions.

In some embodiments, the droplets that are generated are substantially uniform in shape and/or size. For example, in some embodiments, the droplets are substantially uniform in average diameter. In some embodiments, the droplets that are generated have an average diameter of about 0.001 microns, about 0.005 microns, about 0.01 microns, about 0.05 microns, about 0.1 microns, about 0.5 microns, about 1 microns, about 5 microns, about 10 microns, about 20 microns, about 30 microns, about 40 microns, about 50 microns, about 60 microns, about 70 microns, about 80 microns, about 90 microns, about 100 microns, about 150 microns, about 200 microns, about 300 microns, about 400 microns, about 500 microns, about 600 microns, about 700 microns, about 800 microns, about 900 microns, or about 1000 microns. In some embodiments, the droplets that are generated have an average diameter of less than about 1000 microns, less than about 900 microns, less than about 800 microns, less than about 700 microns, less than about 600 microns, less than about 500 microns, less than about 400 microns, less than about 300 microns, less than about 200 microns, less than about 100 microns, less than about 50 microns, or less than about 25 microns. In some embodiments, the droplets that are generated are non-uniform in shape and/or size.

In some embodiments, the droplets that are generated are substantially uniform in volume. For example, in some embodiments, the droplets that are generated have an average volume of about 0.001 nL, about 0.005 nL, about 0.01 nL, about 0.02 nL, about 0.03 nL, about 0.04 nL, about 0.05 nL, about 0.06 nL, about 0.07 nL, about 0.08 nL, about 0.09 nL, about 0.1 nL, about 0.2 nL, about 0.3 nL, about 0.4 nL, about 0.5 nL, about 0.6 nL, about 0.7 nL, about 0.8 nL, about 0.9 nL, about 1 nL, about 1.5 nL, about 2 nL, about 2.5 nL, about 3 nL, about 3.5 nL, about 4 nL, about 4.5 nL, about 5 nL, about 5.5 nL, about 6 nL, about 6.5 nL, about 7 nL, about 7.5 nL, about 8 nL, about 8.5 nL, about 9 nL, about 9.5 nL, about 10 nL, about 11 nL, about 12 nL, about 13 nL, about 14 nL, about 15 nL, about 16 nL, about 17 nL, about 18 nL, about 19 nL, about 20 nL, about 25 nL, about 30 nL, about 35 nL, about 40 nL, about 45 nL, or about 50 nL. In some embodiments, the droplets have an average volume of about 50 picoliters to about 2 nanoliters. In some embodiments, the droplets have an average volume of about 0.5 nanoliters to about 50 nanoliters. In some embodiments, the droplets have an average volume of about 0.5 nanoliters to about 2 nanoliters.

In some embodiments, the amplification reaction is a droplet digital PCR reaction. Methods for performing PCR in droplets are described, for example, in US 2014/0162266, US 2014/0302503, and US 2015/0031034, the contents of each of which is incorporated by reference. In some embodiments, the QX200, QX600, or QX One Droplet Digital PCR (ddPCR) System (Bio-Rad) is used.

In some embodiments, a detection reagent or a detectable label in the partitions can be detected using any of a variety of detector devices. Exemplary detection methods include optical detection (e.g., fluorescence, or chemiluminescence). As a non-limiting example, a fluorescent label can be detected using a detector device equipped with a module to generate excitation light that can be absorbed by a fluorophore, as well as a module to detect light emitted by the fluorophore.

In some embodiments, the detector further comprises handling capabilities for the partitioned samples (e.g., droplets), with individual partitioned samples entering the detector, undergoing detection, and then exiting the detector. In some embodiments, partitioned samples (e.g., droplets) can be detected serially while the partitioned samples are flowing. In some embodiments, partitioned samples (e.g., droplets) are arrayed on a surface and a detector moves relative to the surface, detecting signal(s) at each position containing a single partition. Examples of detectors are provided in WO 2010/036352, the contents of which are incorporated herein by reference. In some embodiments, detectable labels in partitioned samples can be detected serially without flowing the partitioned samples (e.g., using a chamber slide).

Following acquisition of fluorescence detection data, a general purpose computer system (referred to herein as a “host computer”) can be used to store and process the data.

Kits and mixtures useful for practice of the methods are also provided. Kits can be composed, for example, of plastic containers containing a mixture as described herein, optionally with instructions for its use. For example, in some embodiments, a mixture of forward, or forward and reverse primers as described herein are provided that can be mixed by an end user with a RNaseH2 enzyme, probes, polymerase, and/or other reagents as described herein.

In some embodiments, a mixture comprises at least a first forward primer comprising a 3′ target-specific forward sequence and a reverse primer comprising a 3′ target-specific reverse sequence, wherein the forward primer or the reverse primer further comprise a first 5′ universal sequence, and probe nucleic acids comprising (i) a first fluorophore, (ii) a first quencher, (iii) at least 25% (e.g., at least 50%), or at least 10 (e.g., at least 15, 20, 25 or more) nucleotides, or both, of the 5′ universal sequence and (iv) a least one ribonucleotide separating the fluorophore and the quencher. In some embodiments, the mixture comprises a first set of forward and reverse primers as described above, each of the forward primer or reverse primer having an identical 5′ universal sequence but different (e.g., at least 2, 3, 4, 5, 8, 10, 15, 20, or more) 3′ target-specific forward sequence and 3′ target-specific reverse sequences, respectively, such that at least 2, 3, 4, 5, 8, 10, 15, 20, or more different target nucleic acids can be amplified by the primers and detected by the probe nucleic acid.

In some embodiments, the mixture can comprise at least two different separately detectable probes along with two sets of primers: a first set of primers (forward and reverse) that include a first 5′ universal sequence, whose reverse complement is detectable by the first probe, and a second set of primers (forward and reverse) that include a second 5′ universal sequence, whose reverse complement is detectable by the second probe. For each set of primers, either the forward or reverse primers comprise the 5′ universal sequence, but not both. Either or both sets of primers can comprise a plurality of different primers having different 3′ target-specific sequences allowing for different target nucleic acids within the same set of primers.

For example, in some embodiments, the mixture comprises:
(a) a first set of forward primers comprising 3′ target-specific forward sequences and reverse primers comprising 3′ target-specific reverse sequences, wherein the forward primers or the reverse primers of the first set further comprise a first 5′ universal sequence, wherein the first set targets a first plurality of different target nucleic acids, and a first set of the probe nucleic acids comprising (i) a first fluorophore, (ii) a first quencher, (iii) at least 25% (e.g., at least 50%), or at least 10 (e.g., at least 15, 20, 25 or more) nucleotides, or both, of the first 5′ universal sequence and (iv) a least one ribonucleotide separating the fluorophore and the quencher; and
(b) a second set of forward primers comprising 3′ target-specific forward sequences and reverse primers comprising 3′ target-specific reverse sequences, wherein the forward primers or the reverse primers of the second set further comprise a second 5′ universal sequence, wherein the second set targets a second plurality of different target nucleic acids, and a second set of the probe nucleic acids comprising (i) a second fluorophore, (ii) a second quencher, (iii) at least 25% (e.g., at least 50%), or at least 10 (e.g., at least 15, 20, 25 or more) nucleotides, or both, of the second 5′ universal sequence and (iv) a least one ribonucleotide separating the fluorophore and the quencher, such that first amplicons from the first set of forward and reverse primers and having the first 5′ universal sequence can be distinguished from second amplicons from the second set of forward and reverse primers and having the second 5′ universal sequence based on signal from the first set of the probe nucleic acids and the second set of the probe nucleic acids, respectively.

The mixtures can further comprise a third, fourth, fifth, sixth or more sets of primers and probes like those above but targeting different target nucleic acids and having probes that detect different universal sequences as described herein, allowing for higher degrees of multiplexing.

### Example

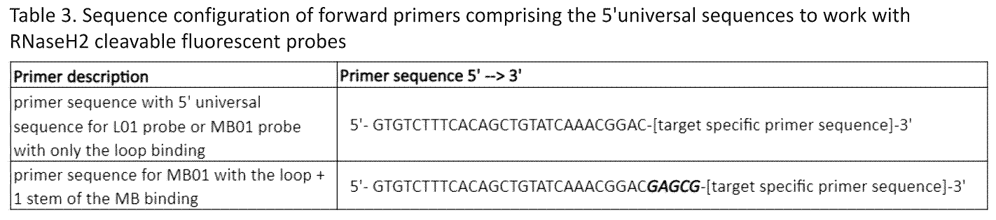

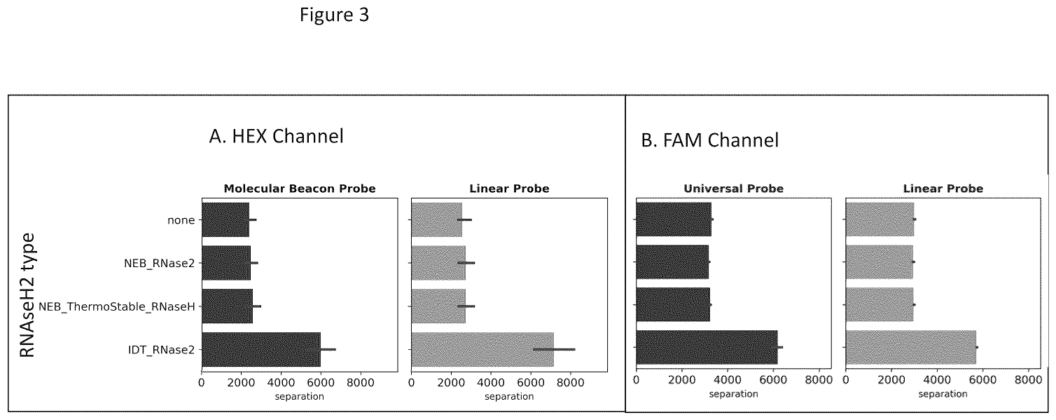

FIG. 3 shows results from a single primer pair with the forward primer comprising the 5′ universal sequence that was tested on the Bio-Rad QX600 Droplet Digital PCR System using a molecular beacon with one RNA base in some reaction wells and a linear probe with an RNA base in other reaction wells. The testing plan evaluated different types of RNase H2 and compared it against the RNA base probe without RNaseH2 added to the reaction.

The 20 μl reactions contain 5 μl of ddPCR supermix, 0.27 μl of DTT, 90 nM of forward primer, 900 nM of reverse primer, 500 nM of fluorescent probe, 0.25 μl of RNase H2, 5 ng of genomic DNA and nuclease free water to adjust final reaction volume to 20 μl. In reaction wells without RNaseH2, 0.25 μl of nuclease free water is added instead. In reaction wells with RNaseH2 the following enzymes and concentrations were tested: NEB RNaseH2 (catalog number M0288S) at 50 mU per reaction, and IDT RNaseH2 (catalog number 11-03-02-02) at 50 mU per reaction. A thermostable RNaseH enzyme was also tested, the NEB thermostable RNaseH (catalog number M0523 S) at 50 mU per reaction. Once ddPCR reactions were set up, droplets were generated with the Bio-Rad AutoDG instrument and droplets were thermocycled on a Bio-Rad C1000 thermocycler using the following cycle: 95° C. for 10 min, 40 cycles of 94° C. for 30 sec and 59° C. for 1 min, final denaturation step at 98° C. for 10 min and cooling down to 4° C. with a ramp rate of 2.5° C./sec. Droplet fluorescence was measured with the Bio-Rad QX600 reader.

The testing was performed with HEX (FIG. 3 Panel A) and FAM (FIG. 3 Panel B) fluorophores. Greater than 2-fold fluorescence increase of the positive droplets was measured for both the molecular beacons and linear probes with RNA base in presence of IDT RNaseH2 compared to the reactions without RNaseH2. Limited to no fluorescence increase was observed with NEB RNaseH2 and NEB thermostable RNaseH.

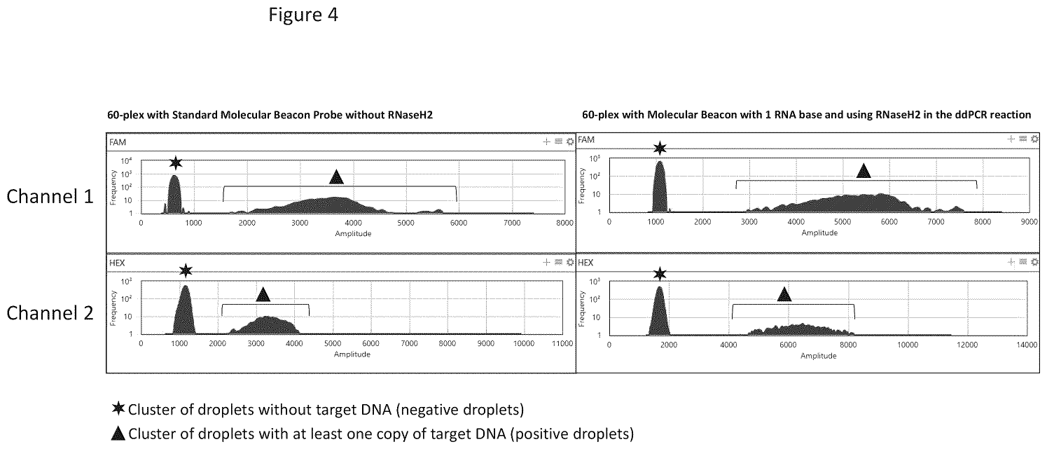

FIG. 4 displays results from a 60-plex tested on the Bio-Rad QX600 Droplet Digital PCR System comparing standard molecular beacons and RNaseH2 cleavable molecular beacons. The 60-plex (shown in the FIG. 3) is composed of 10 primer pairs detecting target 1 in the HEX channel, 10 primer pairs detecting target 2 in the FAM channel, 10 primer pairs detecting target 3 in the Cy5 channel, 10 primer pairs detecting target 4 in the Cy5.5 channel, 10 primer pairs detecting target 5 in the ROX channel, 10 primer pairs detecting target 6 in the Atto590 channel. Within each set of 10 primer pairs, all the forward primers have the same 5′ universal sequences, so they are detected with the same molecular beacon probe. For instance, if MB01 (see Table 1) is used to detect target 1, all 10 forward primers used for Target 1 will have the 5′ universal sequence compatible with MB01 (see Table 3). The same logic applies to each fluorescent channel. In this case, despite having a total of 60 primer pairs in the ddPCR reaction across 6 fluorescent channels, only 6 fluorescent probes are used for detection.

The ddPCR reactions contained 5 μl of ddPCR supermix, 0.27 μl of DTT, 90 nM of each forward primer, 900 nM of each reverse primer, 500 nM of each fluorescent probe, 100 mU of IDT RNaseH2 (catalog number 11-03-02-02), 5 ng of genomic DNA and nuclease free water to adjust final reaction volume to 20 μl. In reaction wells with standard molecular beacons, RNaseH2 was replaced with 0.25 μl of nuclease free water. Once ddPCR reactions were set up, droplets were generated with the Bio-Rad AutoDG instrument and droplets were thermocycled on a Bio-Rad C1000 thermocycler using the following cycle: 95° C. for 10 min, 40 cycles of 94° C. for 30 sec and 59° C. for 1 min, final denaturation step at 98° C. for 10 min and cooling down to 4° C. with a ramp rate of 2.5° C./sec. Droplet fluorescence was measured with the Bio-Rad QX600 reader. The 1D histogram plots show droplet fluorescence amplitude on the X axis versus number of droplets on the Y axis. The peaks labeled with a star correspond to negative droplets with no target DNA. The peaks labeled with a triangle correspond to positive droplets with at least 1 copy of target DNA. The results show that the fluorescence amplitude separation between the negative droplet cluster and the positive droplet cluster increase by more than 2 fold with RNaseH2 cleavable molecular beacons compared to standard molecular beacons. The spread of the positive droplet cluster is also greater in that configuration. This demonstrates the increase in fluorescence signal with the molecular beacons with the RNA base combined with the use of RNaseH2 in the ddPCR reaction compared to standard molecular beacons for high multiplexing applications. A 96-plex with 16 primer pairs per channel across 6 channels, and a 120-plex with 20 primer pairs per channel across 6 channels were tested using the same configuration as explained for the 60-plex (data not shown).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of detecting a target nucleic acid in a sample, the method comprising,
(a) forming a reaction mixture comprising:
sample nucleic acids;
a plurality of forward primers comprising 3′ target-specific forward sequences,
a plurality of reverse primers comprising 3′ target-specific reverse sequences, wherein the forward primers or the reverse primers further comprise a 5′ universal sequence;
probe nucleic acids comprising (i) a fluorophore, (ii) a quencher, (iii) at least 25% (e.g., at least 50%), or at least 10 (e.g., at least 15, 20, 25 or more) nucleotides, or both, of the 5′ universal sequence and (iv) having at least one ribonucleotide separating the fluorophore and the quencher;
a DNA polymerase; and
an RNaseH2 enzyme;
(b) annealing the forward primers to target nucleic acids in the sample nucleic acids and extending with a polymerase the forward primers using the target nucleic acids as a template to form first strand extension products;
(c) annealing the reverse primers to the first strand extension products and extending with the polymerase the reverse primers using the first strand extension products as a template to form second strand extension products, wherein the first strand extension products comprise a reverse complement of the 5′ universal sequence if the reverse primers comprise the 5′ universal sequence and the second strand extension products comprise a reverse complement of the 5′ universal sequence if the forward primers comprise the 5′ universal sequence; and
(d) annealing the probe nucleic acids to the reverse complement of the 5′ universal end sequence and the RNase H2 enzyme cleaves the annealed probe at the ribonucleotide, thereby separating the quencher from the fluorophore to generate a detectable signal indicating the presence of the target nucleic acid.

2. The method of claim 1, wherein (a)-(d) occurs in partitions wherein the target nucleic acid is distributed among the partitions such that at least a portion of the partitions do not contain a target nucleic acid.

3. The method of claim 1, wherein the sample nucleic acids are cell-free DNA.

4. The method of claim 3, wherein the sample nucleic acids are from a pregnant woman and contain maternal and fetal DNA.

5. The method of claim 2, wherein the partitions are droplets.

6. The method of claim 2, wherein the partitions are microwells.

7. The method of claim 1, wherein the forward primers comprise the 5′ universal sequence and the concentration of reverse primers is higher than the concentration of forward primers.

8. The method of claim 1, wherein the reverse primers comprise the 5′ universal sequence and the concentration of forward primers is higher than the concentration of forward primers.

9. The method of claim 1, wherein the forward primers comprise the 5′ universal sequence and the reverse primers have a 5′ tail sequence that does not anneal to the target nucleic acids.

10. The method of claim 1, wherein the reverse primers comprise the 5′ universal sequence and the forward primers have a 5′ tail sequence that does not anneal to the target nucleic acids.

11. The method of claim 1, wherein the probe nucleic acids are linear probes.

12. The method of claim 11, wherein the forward primer comprises the 5′ universal sequence and the linear probes and the reverse complement of the 5′ universal sequence on the second strand extension products form a duplex having a higher melting temperature than a duplex formed from the 5′ universal sequence of the forward primer and the reverse complement of the 5′ universal sequence.

13. The method of claim 11, wherein the reverse primer comprises the 5′ universal sequence and the linear probes and the reverse complement of the 5′ universal sequence on the first strand extension products form a duplex having a higher melting temperature than a duplex formed from the 5′ universal sequence of the reverse primers and the reverse complement of the 5′ universal sequence.

14. The method of claim 1, wherein the probe nucleic acids form a stem-loop and comprise 5′ to 3′: a first stem sequence, a loop sequence, and a second stem sequence that is the reverse complement of the first stem sequence, wherein the ribonucleotide is in the loop sequence, and wherein the 5′ universal sequence comprises at least part of the loop sequence.

15. The method of claim 14, wherein the 5′ universal sequence further comprises at least part of the second stem sequence.

16. The method of claim 14, wherein the 5′ universal sequence comprises all of the loop sequence, all of the second stem sequence, or all of the loop sequence and second stem sequence.

17. The method of claim 1, wherein the RNase H2 enzyme is a Pyrococcus abyssi RNase H2 enzyme or a mutant thereof, Pyrococcus furiosis RNase H2 enzyme or a mutant thereof, Pyrococcus horikoshii RNase H2 enzyme or a mutant thereof, Thermococcus kodakarensis RNase H2 enzyme or a mutant thereof, or a Thermococcus litoralis RNase H2 enzyme or a mutant thereof.

18. The method of claim 1, wherein the DNA polymerase lacks 5′-3′ exonuclease activity.

19. A reaction mixture comprising
a plurality of forward primers comprising 3′ target-specific forward sequences,
a plurality of reverse primers comprising 3′ target-specific reverse sequences, wherein the forward primers or the reverse primers further comprise a 5′ universal sequence;
probe nucleic acids comprising (i) a fluorophore, (ii) a quencher, (iii) at least 25% (e.g., at least 50%), or at least 10 (e.g., at least 15, 20, 25 or more) nucleotides, or both, of the 5′ universal sequence and (iv) having a least one ribonucleotide separating the fluorophore and the quencher;
a DNA polymerase; and
an RNaseH2 enzyme.

20. A mixture comprising,
(a) a first set of forward primers comprising 3′ target-specific forward sequences and reverse primers comprising 3′ target-specific reverse sequences, wherein the forward primers or the reverse primers of the first set further comprise a first 5′ universal sequence, wherein the first set targets a first plurality of different target nucleic acids, and
a first set of the probe nucleic acids comprising (i) a first fluorophore, (ii) a first quencher, (iii) at least 25% (e.g., at least 50%), or at least 10 (e.g., at least 15, 20, 25 or more) nucleotides, or both, of the first 5′ universal sequence and (iv) a least one ribonucleotide separating the fluorophore and the quencher; and
(b) a second set of forward primers comprising 3′ target-specific forward sequences and reverse primers comprising 3′ target-specific reverse sequences, wherein the forward primers or the reverse primers of the second set further comprise a second 5′ universal sequence, wherein the second set targets a second plurality of different target nucleic acids, and
a second set of the probe nucleic acids comprising (i) a second fluorophore, (ii) a second quencher, (iii) at least 25% (e.g., at least 50%), or at least 10 (e.g., at least 15, 20, 25 or more) nucleotides, or both, of the second 5′ universal sequence and (iv) a least one ribonucleotide separating the fluorophore and the quencher,
such that first amplicons from the first set of forward and reverse primers and having the first 5′ universal sequence can be distinguished from second amplicons from the second set of forward and reverse primers and having the second 5′ universal sequence based on signal from the first set of the probe nucleic acids and the second set of the probe nucleic acids, respectively.
